(19) United States
(12) Patent Application Publication
(10) Pub. No.: US 2025/0120744 A1
(43) Pub. Date: Apr. 17, 2025

(54) SPERM EXTRACTOR WITH DOUBLE FOLDING HANDLES

(71) Applicant: Zhejiang lianhong Technology Co., LTD, Huzhou (CN)

(72) Inventor: Xiu CHEN, Hangzhou (CN)

(73) Assignee: Zhejiang lianhong Technology Co., LTD, Huzhou (CN)

(21) Appl. No.: 18/769,903

(22) Filed: Jul. 11, 2024

(30) Foreign Application Priority Data
Oct. 13, 2023 (CN) 2023227702233

(51) Int. Cl. A61B 17/43; A61B 17/00

(52) U.S. Cl. CPC A61B 17/43; A61B 2017/00199; A61B 2017/00398; A61B 2017/00424; A61B 2017/0046; A61B 2017/00734; A61B 2017/00876

(57) ABSTRACT

Provided in the present utility model is a sperm extractor with double folding handles, including: a housing; the double folding handles mounted on the housing by mounting brackets; and an inner container mounted inside the housing, where the double folding handles include a left handle and a right handle that are disposed on a left side and a right side of the housing respectively; the mounting brackets include a first mounting bracket and a second mounting bracket that are disposed on an upper side and a lower side of the housing respectively.

### CROSS-REFERENCE TO RELATED APPLICATIONS

The application claims priority to Chinese patent application No. 2023227702233, filed on Oct. 13, 2023, the entire contents of which are incorporated herein by reference.

### TECHNICAL FIELD

The present utility model relates to the technical field of medical devices, specifically to a sperm extractor with double folding handles.

### BACKGROUND

With the continuous development of economy and society, people's life pressure is also increasing, leading to a decrease in male sperm motility and an increasingly prominent problem of infertility. During clinic services in existing hospitals, it is often required to extract semen in a short time and conduct various tests on the semen.

In the prior art, a sperm extractor is provided with a handheld structure, making it prone to falling damage.

Therefore, the prior art has shortcomings and needs to be further improved.

### SUMMARY

In view of the problems existing in the prior art, the present utility model provides a sperm extractor with double folding handles.

To achieve the above objective, a specific solution of the present utility model is as follows:

The present utility model provides a sperm extractor with double folding handles, including:
a housing;
the double folding handles mounted on the housing by mounting brackets; and
an inner container mounted inside the housing,
where the double folding handles include a left handle and a right handle that are disposed on a left side and a right side of the housing respectively.

Further, the mounting brackets include a first mounting bracket and a second mounting bracket that are disposed on an upper side and a lower side of the housing respectively;
an upper end and a lower end of the left handle are movably mounted at a left end of the first mounting bracket and a left end of the second mounting bracket respectively; and
an upper end and a lower end of the right handle are movably mounted at a right end of the first mounting bracket and a right end of the second mounting bracket respectively.

Further, the mounting brackets include a third mounting bracket and a fourth mounting bracket that are disposed on a left side and a right side of the housing respectively; and
the left handle and the right handle are movably mounted on the third mounting bracket and the fourth mounting bracket respectively.

Further, rotating assemblies are disposed at joints of the double folding handles and the mounting brackets;
each of the rotating assemblies includes an inner clamping element and an outer clamping element; and
clamping teeth are disposed on an edge of the inner clamping element, clamping grooves are formed on an inner side of the outer clamping element, the clamping teeth are located in the clamping grooves, the number of the clamping grooves is greater than the number of the clamping teeth,
the inner clamping elements and the outer clamping elements are mounted on the mounting brackets and the double folding handles respectively, and the double folding handles are unfolded or folded on a sidewall of the housing by the inner clamping elements and the outer clamping elements.

Further, a left inner housing and a right inner housing are further disposed on an inner side of the housing; and
the left inner housing and the right inner housing are mutually fastened to form an accommodating space, and the inner container is located in the accommodating space.

Further, a printed circuit board (PCB) and a battery are further disposed on the left inner housing and the right inner housing;
a charging interface is disposed on a lower side of the housing; and
the PCB is electrically connected to the battery and the charging interface separately.

Further, a first power mechanism is further mounted on the inner container;
the first power mechanism includes a first motor, and the first motor is electrically connected to the PCB;
a gearbox is mounted on the first motor, a crank is mounted on an output shaft of the gearbox, a sliding ring is mounted at a front end of the crank, the sliding ring is disposed in a sliding groove of a connecting block, the other end of the connecting block is mounted at a bottom of a sleeve, and a first magnet is disposed on the sleeve;
a first sliding rod and a second sliding rod are disposed on two sides of the sleeve respectively, the sleeve is movably mounted on the first sliding rod and the second sliding rod, and the first sliding rod and the second sliding rod are fixedly mounted on the left inner housing and the right inner housing;
a second magnet is disposed at a tail of the inner container, and the inner container is butted with the sleeve by a magnetic force of the second magnet and the first magnet; and
the first motor drives the connecting block by the crank to drive the sleeve to move back and forth, and the sleeve drives the inner container to move back and forth.

Further, a second power mechanism is further disposed at an opening of the inner container;
the second power mechanism includes a second motor, and the second motor is electrically connected to the PCB;
an eccentric wheel is disposed on a rotating shaft of the second motor; and the second motor drives the eccentric wheel to rotate to generate a vibration.

Further, a display screen and a first button are disposed on the upper side and the lower side of the housing respectively;
a second button and a third button are disposed on the left handle and the right handle respectively; and
the PCB is electrically connected to the display screen, the first button, the second button, and the third button separately.

The present utility model further provides a sperm extractor with double folding handles, including:
a housing;
the double folding handles mounted on the housing by mounting brackets; and
an inner container mounted inside the housing,
where the double folding handles include a left handle and a right handle that are disposed on a left side and a right side of the housing respectively;
two clamping hooks are further disposed at an opening end of the inner container;
a left inner housing and a right inner housing fastened to each other are further disposed in the housing; and
clamping blocks are disposed at positions of the left inner housing and the right inner housing corresponding to the clamping hooks, a gap is formed in each of the left inner housing and the right inner housing, and the clamping hooks enter from the gaps and then are clamped on the clamping blocks through rotation to fix the inner container.

Further, a contact plate is further disposed on a lower side of the opening end of the inner container;
two arc-shaped grooves are further formed in the contact plate;
a second motor is further disposed on the contact plate;
an eccentric wheel is disposed on a rotating shaft of the second motor; and the second motor drives the eccentric wheel to rotate to generate a vibration.

The present utility model further provides a sperm extractor with double folding handles, including:
a housing;
the double folding handles mounted on the housing by mounting brackets;
an inner container mounted inside the housing,
where the double folding handles comprise a left handle and a right handle that are disposed on a left side and a right side of the housing respectively; and
a first motor, where the first motor is mounted inside the housing, a gearbox is mounted on the first motor, a crank is mounted on an output shaft of the gearbox, a front end of the crank is rotatably connected to a connecting rod, a first rotating shaft is disposed at a bottom of a sleeve, the other end of the connecting rod is rotatably connected to the first rotating shaft, the first motor drives the crank by the gearbox to rotate so as to drive the connecting rod to push and pull the sleeve to move back and forth, and the inner container is disposed in the sleeve and moves together with the sleeve.

Further, a first magnet is disposed at the bottom of the sleeve, a second magnet is disposed at a tail of the inner container, the first magnet and the second magnet magnetically attract each other, and the sleeve drives the inner container by a magnetic force to move back and forth.

With the technical solution of the present utility model, the following beneficial effects are achieved:
1. The double folding handles are mounted to avoid falling damage of the sperm extractor, and are foldable to save space.
2. The rotating assemblies limit the left handle and the right handle by the clamping teeth and the clamping grooves, such that the left handle and the right handle can be unfolded or folded on the sidewall of the housing.
3. Telescopic motion and vibration are generated by double motors, which can achieve a better usage effect.
4. Differing from the power mechanism disposed in a middle of the soft rubber inner container to provide a vibration massage in the prior art, the power mechanism is disposed at the opening portion of the soft rubber inner container, which can achieve a better effect.
5. The contact plate is disposed at the opening portion, and the arc-shaped grooves are formed in the contact plate to further fit the skin of the male genital organ, thereby providing a better usage experience.
6. The design of the clamping hooks and the clamping blocks facilitates replacement of the inner container.
7. By introducing the connecting rod, a rotational motion of the motor is effectively converted into a rectilinear push-pull motion of the connecting rod, thereby improving the efficiency of energy transfer. The sperm extractor can implement a reciprocating motion of the sleeve in a relatively simple and compact design that reduces the number of moving parts and makes a device simpler and easier to operate and carry.
8. The inner container and the sleeve are assembled and positioned by the magnetic force between the first magnet and the second magnet, which facilitates quick disassembly and cleaning as well as update and replacement of the inner container, and improves the maintainability and sanitary level of the device.

### BRIEF DESCRIPTION OF DRAWINGS

FIG. 1 is a three-dimensional top view of the present utility model;

FIG. 2 is a three-dimensional bottom view of the present utility model;

FIG. 3 is an exploded view of the present utility model;

FIG. 4 is a three-dimensional view of a rotating assembly in the present utility model;

FIG. 5 is a three-dimensional view of a first power mechanism and a second power mechanism in the present utility model; and

FIG. 6 is an exploded view of a first power mechanism and a second power mechanism in the present utility model.

FIG. 7 is a three-dimensional view of an inner container in the present utility model;

FIG. 8 is a three-dimensional view of an inner container in the present utility model from another perspective;

FIG. 9 is a sectional view of an inner container in the present utility model;

FIG. 10 is a three-dimensional view in which an inner container is connected to a right inner housing by clamping hooks in the present utility model;

FIG. 11 is a three-dimensional view of a left inner housing and a right inner housing in the present utility model; and

FIG. 12 is a schematic structural diagram in which a telescopic motion is implemented by a connecting rod in the present utility model.

### IN THE DRAWINGS

1. housing; 101. display screen; 102. first button;
2. inner container; 201. second magnet;
202. clamping hook; 203. clamping block; 204. gap;
205. contact plate; 2051. arc-shaped groove;
301. left handle; 302. first mounting bracket; 303. second button;
401. right handle; 402. second mounting bracket; 403. third button;
5. rotating assembly; 501. inner clamping element; 5011. clamping tooth; 502. outer clamping element; 5021. clamping groove;
601. left inner housing; 602. right inner housing;
603. PCB; 604. battery; 605. charging interface;
701. first motor; 702. gearbox; 703. crank; 704. sliding ring; 705. connecting block; 7051. sliding groove; 706. sleeve; 707. first magnet; 708. first sliding rod; 709. second sliding rod;
710. connecting rod; 711. first rotating shaft;
801. second motor; and 802. eccentric wheel.

### DETAILED DESCRIPTION OF THE EMBODIMENTS

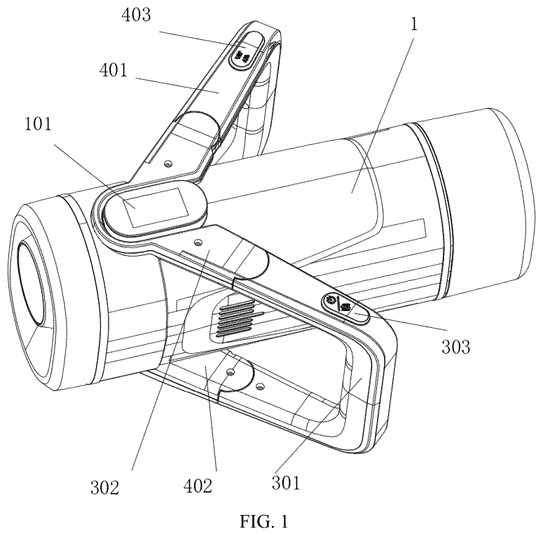

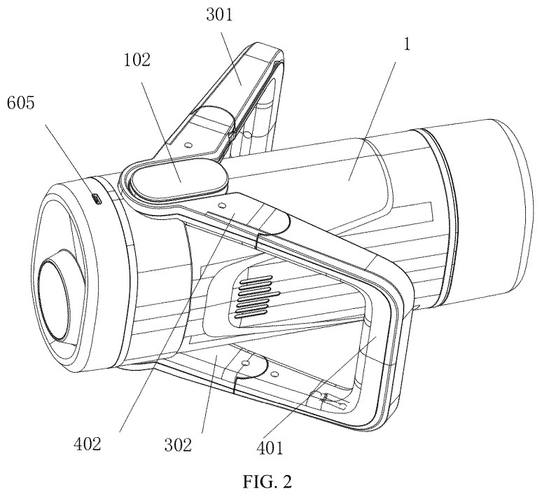

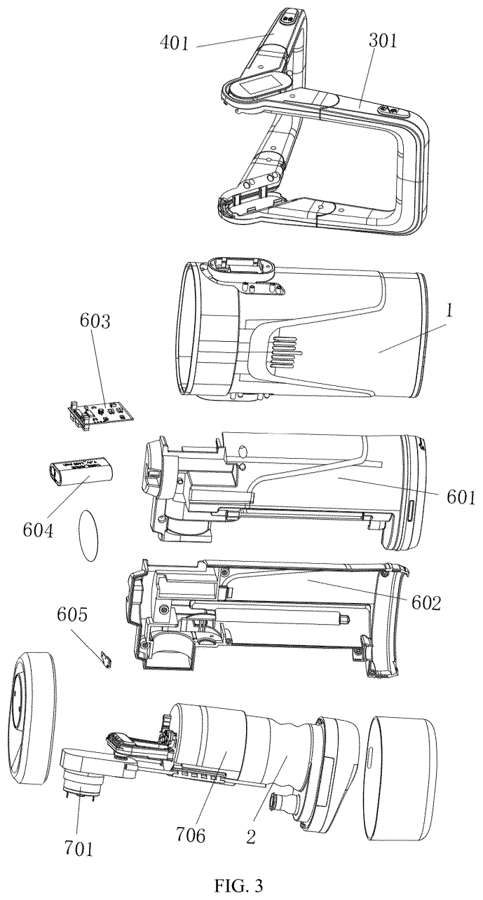

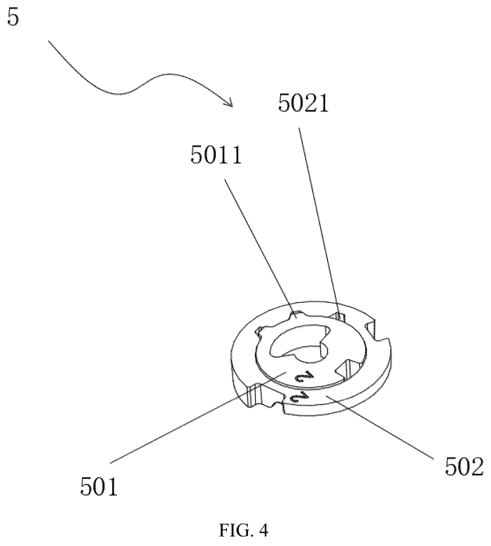

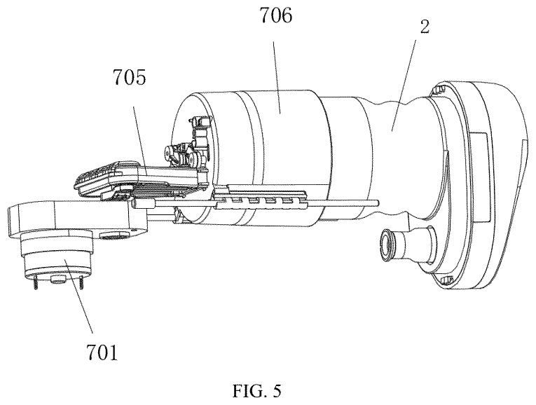

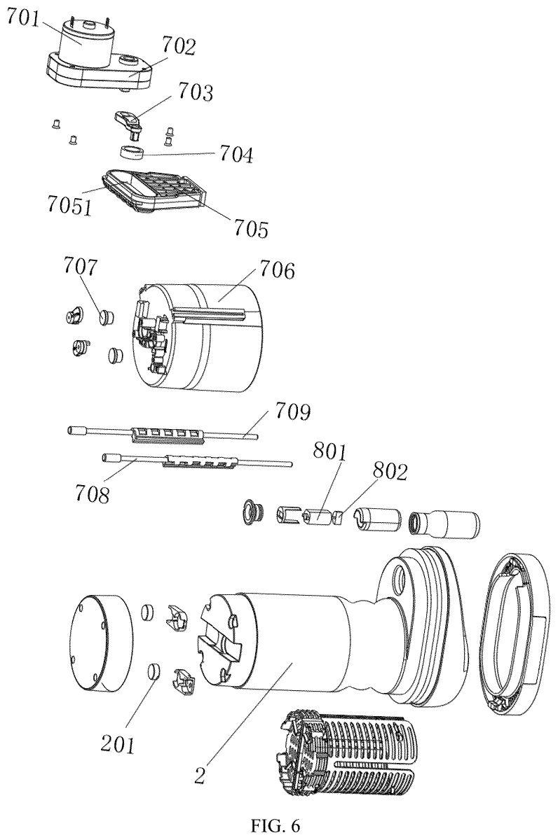

As shown in FIGS. 1 to 6, the present utility model provides a sperm extractor with double folding handles, including: a housing 1; the double folding handles mounted on the housing 1 by mounting brackets; and an inner container 2 mounted inside the housing 1, where the double folding handles include a left handle 3 and a right handle 4 that are disposed on a left side and a right side of the housing 1 respectively.

The mounting brackets include a first mounting bracket 302 and a second mounting bracket 402 that are disposed on an upper side and a lower side of the housing respectively; an upper end and a lower end of the left handle 301 are movably mounted at a left end of the first mounting bracket 302 and a left end of the second mounting bracket 402 respectively; and an upper end and a lower end of the right handle 401 are movably mounted at a right end of the first mounting bracket 302 and a right end of the second mounting bracket 402 respectively.

Rotating assemblies 5 are disposed at joints of the double folding handles and the mounting brackets;
each of the rotating assemblies 5 includes an inner clamping element 501 and an outer clamping element 502; and clamping teeth 5011 are disposed on an edge of the inner clamping element 501, clamping grooves 5021 are formed on an inner side of the outer clamping element 502, the clamping teeth 5011 are located in the clamping grooves 5021, the number of the clamping grooves 5021 is greater than the number of the clamping teeth 5011, the inner clamping elements 501 and the outer clamping elements 502 are mounted on the mounting brackets and the double folding handles respectively, and the double folding handles are unfolded or folded on a sidewall of the housing 1 by the inner clamping elements 501 and the outer clamping elements 502.

A left inner housing 601 and a right inner housing 602 are further disposed on an inner side of the housing 1; the left inner housing 601 and the right inner housing 602 are mutually fastened to form an accommodating space, and the inner container 2 is located in the accommodating space. A PCB 603 and a battery 604 are further disposed on the left inner housing 601 and the right inner housing 602; a charging interface 605 is disposed on a lower side of the housing 1; and the PCB 603 is electrically connected to the battery 604 and the charging interface 605 separately.
a first power mechanism is further mounted on the inner container 2; the first power mechanism includes a first motor 701, and the first motor 701 is electrically connected to the PCB 603; a gearbox 702 is mounted on the first motor 701, a crank 703 is mounted on an output shaft of the gearbox 702, a sliding ring 704 is mounted at a front end of the crank 703, the sliding ring 704 is disposed in a sliding groove 7051 of a connecting block 705, the other end of the connecting block 705 is mounted at a bottom of a sleeve 706, and a first magnet 707 is disposed on the sleeve 706; a first sliding rod 708 and a second sliding rod 709 are disposed on two sides of the sleeve 706 respectively, the sleeve 706 is movably mounted on the first sliding rod 708 and the second sliding rod 709, and the first sliding rod 708 and the second sliding rod 709 are fixedly mounted on the left inner housing 601 and the right inner housing 602; a second magnet 201 is disposed at a tail of the inner container 2, and the inner container 2 is butted with the sleeve 706 by a magnetic force of the second magnet 201 and the first magnet 707; and the first motor 701 drives the connecting block 705 by the crank 703 to drive the sleeve 706 to move back and forth, and the sleeve 706 drives the inner container 2 to move back and forth.

A second power mechanism is further disposed at an opening of the inner container 2; the second power mechanism includes a second motor 801, and the second motor 801 is electrically connected to the PCB 603; an eccentric wheel 802 is disposed on a rotating shaft of the second motor 801; and the second motor 801 drives the eccentric wheel 802 to rotate to generate a vibration.

A display screen 101 and a first button 102 are disposed on the upper side and the lower side of the housing 1 respectively; a second button 304 and a third button 404 are disposed on the left handle 3 and the right handle 4 respectively; and the PCB 603 is electrically connected to the display screen 101, the first button 102, the second button 304, and the third button 404 separately.

Another embodiment of the present application is as follows:

The mounting brackets include a third mounting bracket and a fourth mounting bracket that are disposed on a left side and a right side of the housing respectively; and the left handle 301 and the right handle 401 are movably mounted on the third mounting bracket and the fourth mounting bracket respectively.

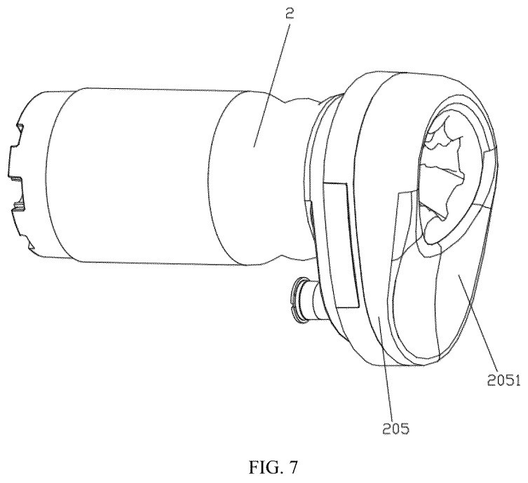

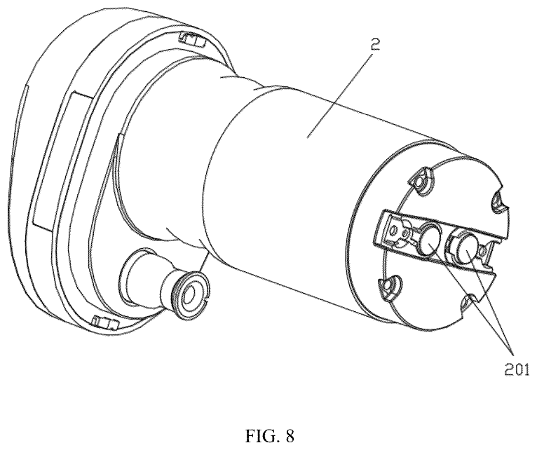

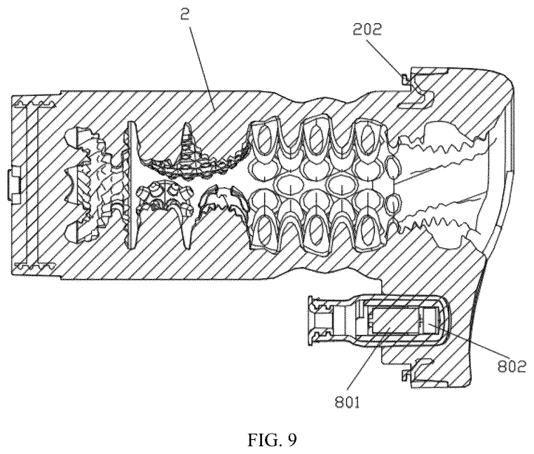

As shown in FIGS. 7 to 9,
a contact plate 205 is further disposed on a lower side of an opening end of the inner container 2;
two arc-shaped grooves 2051 are further formed in the contact plate 205; a second motor 801 is further disposed on the contact plate 205;
an eccentric wheel 802 is disposed on a rotating shaft of the second motor 801; and the second motor 801 drives the eccentric wheel 802 to rotate to generate a vibration.

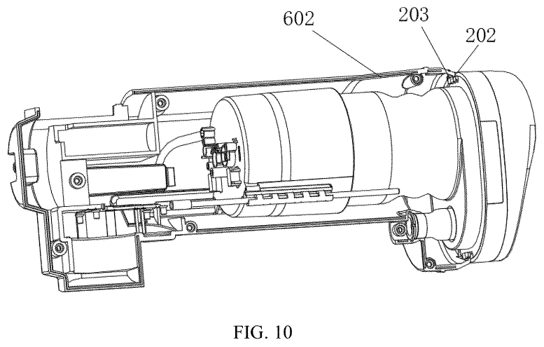

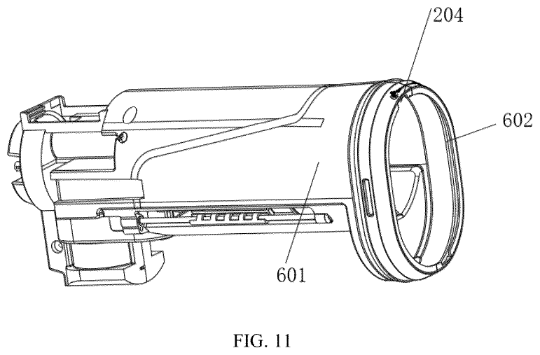

As shown in FIGS. 10 and 11,
two clamping hooks 202 are further disposed at the opening end of the inner container 2;
a left inner housing 601 and a right inner housing 602 fastened to each other are further disposed in the housing 1; and
clamping blocks 203 are disposed at positions of the left inner housing 601 and the right inner housing 602 corresponding to the clamping hooks 202, a gap 204 is formed in each of the left inner housing 601 and the right inner housing 602, and the clamping hooks 202 enter from the gaps 204 and then are clamped on the clamping blocks 203 through rotation to fix the inner container 2.

Yet another embodiment of the present application is as follows:

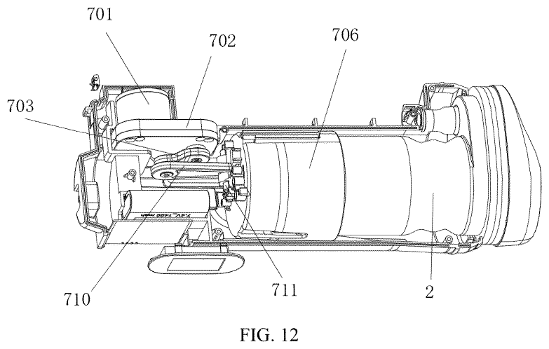

As shown in FIG. 12,
a first motor 701 is mounted inside the housing 1, a gearbox 702 is mounted on the first motor 701, a crank 703 is mounted on an output shaft of the gearbox 702, a front end of the crank 703 is rotatably connected to a connecting rod 710, a first rotating shaft 711 is disposed at a bottom of a sleeve 706, the other end of the connecting rod 710 is rotatably connected to the first rotating shaft 711, the first motor 701 drives the crank 703 by the gearbox 702 to rotate so as to drive the connecting rod 710 to push and pull the sleeve 706 to move back and forth, and the inner container 2 is disposed in the sleeve 706 and moves together with the sleeve 706; and a first magnet 707 is disposed at the bottom of the sleeve 706, a second magnet 201 is disposed at a tail of the inner container 2, the first magnet 707 and the second magnet 201 magnetically attract each other, and the sleeve 706 drives the inner container 2 by a magnetic force to move back and forth. When the inner container 2 is inserted into the sleeve 706 and approaches the bottom of the sleeve 706, the inner container 2 and the sleeve 706 are assembled and positioned by a magnetic force between the first magnet 707 and the second magnet 201, which facilitates quick disassembly and cleaning as well as update and replacement of the inner container 2, and improves the maintainability and sanitary level of a device.

The principle of the present utility model is as follows:

The double folding handles include a left handle 3 and a right handle 4 movably mounted on a left side and a right side of the housing 1. rotating assemblies 5 are disposed at joints of the double folding handles and the mounting brackets; each of the rotating assemblies 5 includes an inner clamping element 501 and an outer clamping element 502; and clamping teeth 5011 are disposed on an edge of the inner clamping element 501, clamping grooves 5021 are formed on an inner side of the outer clamping element 502, the clamping teeth 5011 are located in the clamping grooves 5021, the number of the clamping grooves 5021 is greater than the number of the clamping teeth 5011, the inner clamping elements 501 and the outer clamping elements 502 are mounted on the mounting brackets and the double folding handles respectively, and the double folding handles are unfolded or folded on a sidewall of the housing 1 by the inner clamping elements 501 and the outer clamping elements 502.

The above are only preferred embodiments of the present utility model and are not intended to limit the scope of patent of the present utility model. Any equivalent structural changes made using the contents of the specification and drawings of the present utility model under the concept of the present utility model, directly/indirectly applied in other related technical fields, are included within the scope of patent protection of the present utility model.

What is claimed is:

1. A sperm extractor with double folding handles, comprising:
a housing;
the double folding handles mounted on the housing by mounting brackets; and
an inner container mounted inside the housing,
wherein the double folding handles comprise a left handle and a right handle that are disposed on a left side and a right side of the housing respectively.

2. The sperm extractor with double folding handles according to claim 1, wherein
the mounting brackets comprise a first mounting bracket and a second mounting bracket that are disposed on an upper side and a lower side of the housing respectively;
an upper end and a lower end of the left handle are movably mounted at a left end of the first mounting bracket and a left end of the second mounting bracket respectively; and
an upper end and a lower end of the right handle are movably mounted at a right end of the first mounting bracket and a right end of the second mounting bracket respectively.

3. The sperm extractor with double folding handles according to claim 1, wherein
the mounting brackets comprise a third mounting bracket and a fourth mounting bracket that are disposed on a left side and a right side of the housing respectively; and
the left handle and the right handle are movably mounted on the third mounting bracket and the fourth mounting bracket respectively.

4. The sperm extractor with double folding handles according to claim 1, wherein
rotating assemblies are disposed at joints of the double folding handles and the mounting brackets;
each of the rotating assemblies comprises an inner clamping element and an outer clamping element; and
clamping teeth are disposed on an edge of the inner clamping element, clamping grooves are formed on an inner side of the outer clamping element, the clamping teeth are located in the clamping grooves, the number of the clamping grooves is greater than the number of the clamping teeth,
the inner clamping elements and the outer clamping elements are mounted on the mounting brackets and the double folding handles respectively, and the double folding handles are unfolded or folded on a sidewall of the housing by the inner clamping elements and the outer clamping elements.

5. The sperm extractor with double folding handles according to claim 1, wherein
a left inner housing and a right inner housing are further disposed on an inner side of the housing; and
the left inner housing and the right inner housing are mutually fastened to form an accommodating space, and the inner container is located in the accommodating space.

6. The sperm extractor with double folding handles according to claim 5, wherein
a printed circuit board (PCB) and a battery are further disposed on the left inner housing and the right inner housing;
a charging interface is disposed on a lower side of the housing; and
the PCB is electrically connected to the battery and the charging interface separately.

7. The sperm extractor with double folding handles according to claim 6, wherein
a first power mechanism is further mounted on the inner container;
the first power mechanism comprises a first motor, and the first motor is electrically connected to the PCB;
a gearbox is mounted on the first motor, a crank is mounted on an output shaft of the gearbox, a sliding ring is mounted at a front end of the crank, the sliding ring is disposed in a sliding groove of a connecting block, the other end of the connecting block is mounted at a bottom of a sleeve, and a first magnet is disposed on the sleeve;
a first sliding rod and a second sliding rod are disposed on two sides of the sleeve respectively, the sleeve is movably mounted on the first sliding rod and the second sliding rod, and the first sliding rod and the second sliding rod are fixedly mounted on the left inner housing and the right inner housing;
a second magnet is disposed at a tail of the inner container, and the inner container is butted with the sleeve by a magnetic force of the second magnet and the first magnet; and
the first motor drives the connecting block by the crank to drive the sleeve to move back and forth, and the sleeve drives the inner container to move back and forth.

8. The sperm extractor with double folding handles according to claim 7, wherein
a second power mechanism is further disposed at an opening of the inner container;
the second power mechanism comprises a second motor, and the second motor is electrically connected to the PCB;
an eccentric wheel is disposed on a rotating shaft of the second motor; and the second motor drives the eccentric wheel to rotate to generate a vibration.

9. The sperm extractor with double folding handles according to claim 8, wherein
a display screen and a first button are disposed on the upper side and the lower side of the housing respectively;
a second button and a third button are disposed on the left handle and the right handle respectively; and
the PCB is electrically connected to the display screen, the first button, the second button, and the third button separately.

10. A sperm extractor with double folding handles, comprising:
a housing;
the double folding handles mounted on the housing by mounting brackets; and
an inner container mounted inside the housing,
wherein the double folding handles comprise a left handle and a right handle that are disposed on a left side and a right side of the housing respectively;
two clamping hooks are further disposed at an opening end of the inner container;
a left inner housing and a right inner housing fastened to each other are further disposed in the housing; and
clamping blocks are disposed at positions of the left inner housing and the right inner housing corresponding to the clamping hooks, a gap is formed in each of the left inner housing and the right inner housing, and the clamping hooks enter from the gaps and then are clamped on the clamping blocks through rotation to fix the inner container.

11. The sperm extractor with double folding handles according to claim 10, wherein
a contact plate is further disposed on a lower side of the opening end of the inner container;
two arc-shaped grooves are further formed in the contact plate;
a second motor is further disposed on the contact plate;
an eccentric wheel is disposed on a rotating shaft of the second motor; and the second motor drives the eccentric wheel to rotate to generate a vibration.

12. A sperm extractor with double folding handles, comprising:
a housing;
the double folding handles mounted on the housing by mounting brackets;
an inner container mounted inside the housing,
wherein the double folding handles comprise a left handle and a right handle that are disposed on a left side and a right side of the housing respectively; and
a first motor, wherein the first motor is mounted inside the housing, a gearbox is mounted on the first motor, a crank is mounted on an output shaft of the gearbox, a front end of the crank is rotatably connected to a connecting rod, a first rotating shaft is disposed at a bottom of a sleeve, the other end of the connecting rod is rotatably connected to the first rotating shaft, the first motor drives the crank by the gearbox to rotate so as to drive the connecting rod to push and pull the sleeve to move back and forth, and the inner container is disposed in the sleeve and moves together with the sleeve.

13. The sperm extractor with double folding handles according to claim 12, wherein
a first magnet is disposed at the bottom of the sleeve, a second magnet is disposed at a tail of the inner container, the first magnet and the second magnet magnetically attract each other, and the sleeve drives the inner container by a magnetic force to move back and forth.
